(19) United States
(12) Patent Application Publication
(10) Pub. No.: US 2019/0125445 A1
(43) Pub. Date: May 2, 2019

(54) SYSTEMS AND METHODS FOR FORMING COMPLEX TREATMENT PROFILES IN SKIN

(71) Applicant: Sciton, Inc., Palo Alto, CA (US)

(72) Inventors: Frank GARCIA, Redwood City, CA (US); David BOINAGROV, Mountain View, CA (US); Hartmuth HECHT, Moss Beach, CA (US); James L. HOBART, Palo Alto, CA (US); Daniel K. NEGUS, Palo Alto, CA (US)

(21) Appl. No.: 16/089,518

(22) PCT Filed: Apr. 7, 2017
(86) PCT No.: PCT/US17/26586

§ 371 Date: Sep. 28, 2018

Related U.S. Application Data
(60) Provisional application No. 62/320,355, filed on Apr. 8, 2016.

(51) Int. Cl. A61B 18/20; A61N 5/06

(52) U.S. Cl. CPC A61B 18/203; A61B 2018/00589; A61N 5/0616

(57) ABSTRACT

In one aspect, methods of laser treatment of skin are described herein. In some embodiments, such a method comprises forming at least one fractional column or region of tissue in skin of a patient, wherein the fractional column has a structure along a z-direction orthogonal to an exterior surface of the skin, and wherein the structure of the fractional column varies along the z-direction in one or more ways. For instance, the structure of the fractional column can vary along the z-direction in one or more of the following ways: angular orientation relative to the exterior surface of the skin; ablated channel width or diameter; coagulation zone thickness; coagulation zone offset in an x-direction or a y-direction perpendicular to the z-direction; coagulation zone intensity; and thermal insult. Moreover, in some cases, the fractional column is defined by a plurality of segments that differ in a manner described above.

### CROSS REFERENCE TO RELATED APPLICATIONS

This application claims priority pursuant to 35 U.S.C. § 119 to U.S. Provisional Patent Application Ser. No. 62/320,355, filed on Apr. 8, 2016, which is hereby incorporated by reference in its entirety.

### FIELD

This invention relates to systems and methods for laser-assisted treatment of patients and, in particular, to systems and methods for forming complex treatment profiles in the skin of patients, such as for drug delivery, thermal shock, or other treatment.

### BACKGROUND

Lasers can be used to treat patients, or assist in the treatment of patients, in a variety of ways. For instance, lasers can be used to assist in drug delivery to a patient. In transdermal drug delivery, one goal is to reduce the highly effective barrier to drug uptake imposed by the stratum corneum (SC). For example, the absorption of topical medicines through an unmodified SC is limited to the range of 1-5%. With the rise in cost of therapeutic substances, particularly in the category of cancer medication, such low absorption rates are not ideal.

Unfortunately, some previous approaches to improving transdermal drug delivery—such as so-called biomodulation, removal of the SC, or aggressive perforation of the skin—suffer from one or more disadvantages. See, e.g., U.S. Pat. Nos. 4,775,361, 6,315,772, and 8,968,221. In fractional photothermolysis (described generally in Manstein et al., Lasers in Surgery and Medicine 34:426-438 (2004)), arrays of ablated channels in skin can be created. However, previous methods of performing fractional photothermolysis have failed to provide structures or pathways needed to achieve substantial improvements in laser-assisted drug delivery, including cutaneous drug delivery.

Thus, there is a need for improved systems and methods for laser-assisted treatment of patients, including for drug delivery.

### SUMMARY

Systems and methods for treating patients or skin of patients are described herein which, in some cases, can provide one or more advantages compared to some other systems and methods. For example, in some embodiments, a system or method described herein can improve drug delivery to a patient by forming a complex fractional structure in a patient that is configured to match or correspond to a diffusion profile or other delivery profile of the drug, which may be applied topically to the skin of the patient on or near the complex fractional structure. Similarly, a system or method described herein, in some instances, can be used to create a complex thermal insult profile in skin of a patient, including in a manner calculated to provide superior therapeutic effect, as compared to monolithic thermal insult profiles.

In one aspect, methods of laser treatment are described herein. In some embodiments, such a method comprises forming at least one fractional column or region of tissue in skin of a patient, wherein the fractional column has a structure along a z-direction orthogonal to an exterior surface of the skin, and wherein the structure of the fractional column varies along the z-direction in one or more ways. More particularly, in some cases, the structure of the fractional column varies along the z-direction in one or more of the following ways: angular orientation relative to the exterior surface of the skin; ablated channel width or diameter; coagulation zone thickness; coagulation zone offset in an x-direction or a y-direction perpendicular to the z-direction; coagulation zone intensity; and thermal insult.

Further, in some embodiments, the structure of the fractional column along the z-direction is defined by a plurality of segments (“stacked” in the z-direction) that differ in one or more of the foregoing ways. For instance, the plurality of segments can differ in one or more of angular orientation relative to the exterior surface of the skin, ablated channel width, coagulation zone thickness, coagulation zone offset in an x-direction or a y-direction perpendicular to the z-direction, coagulation zone intensity, and thermal insult. Moreover, as described further hereinbelow, the segments can vary in a continuous or discontinuous manner. In some cases, for example, the plurality of segments define a step function with respect to one or more of the angular orientation relative to the exterior surface of the skin, ablated channel width, coagulation zone thickness, coagulation zone offset in an x-direction or a y-direction perpendicular to the z-direction, coagulation zone intensity, and thermal insult. Alternatively, in other instances, the plurality of segments define a continuous function with respect to one or more of the angular orientation relative to the exterior surface of the skin, ablated channel width, coagulation zone thickness, coagulation zone offset in an x-direction or a y-direction perpendicular to the z-direction, coagulation zone intensity, and thermal insult. Structures such as those described above can exhibit complex molecular (e.g., drug) diffusion profiles and/or thermal insult profiles. In some cases, for example, at least two segments of the plurality of segments have different molecular diffusion rates and/or different temperature gradients in a lateral direction orthogonal to the z-direction.

It is further to be understood that, in some embodiments, forming at least one fractional column according to a method described herein comprises, or is carried out by, applying a plurality of doses of fractionally ablative laser light to the skin of the patient. Moreover, forming the at least one fractional column can also comprise, or be carried out by, applying one or more doses of non-ablative coagulative laser light to the skin of the patient. Thus, in some instances, forming a fractional column comprises or is carried out by applying a plurality of doses of fractionally ablative laser light and a plurality of doses of non-ablative coagulative laser light to a first spot or location on the skin of the patient, thereby forming the fractional column having a complex structure, as described in further detail below.

Moreover, methods described herein are not limited to forming only a single fractional column or region having a complex structure that varies as a function of the z-direction. Instead, in some cases, a method described herein comprises forming a plurality of fractional columns of tissue in the skin of the patient, including in a manner described hereinabove. Additionally, in some such instances, the plurality of fractional columns defines an array in an xy-plane defined by the exterior surface of the skin of the patient. In some embodiments, the fractional columns are formed substantially simultaneously. In other cases, the fractional columns are formed non-simultaneously or sequentially.

In addition, in some embodiments, a method described herein further comprises imaging a treatment area of the skin of the patient to obtain an image of the treatment area. Moreover, in some cases, forming one or more fractional columns in the skin of the patient comprises forming the one or more fractional columns within the treatment area of the skin of the patient, and imaging the treatment area is carried out before forming the one or more fractional columns in the skin of the patient. Further, in some such instances, the method also comprises diagnosing a condition (or a disease, malady, disorder, or treatment modality) of the skin of the patient based on the image of the treatment area. Additionally, this diagnosis can occur before forming the one or more fractional columns in the skin of the patient. For example, in some embodiments, a method described herein further comprises determining one or more features of at least one fractional column based on the diagnosed condition of the skin, before forming the fractional column in the skin of the patient. Thus, a method described herein can be a “smart” method in which one or more specific, complex fractional structures are formed in skin based on image-based diagnoses of skin conditions. Additionally, such image-based diagnoses may be automated.

Further, methods described herein, in some cases, also comprise applying a pharmaceutical composition or drug to the exterior surface of the skin. In some such embodiments, the pharmaceutical composition or drug is applied to the exterior surface of the skin in a treatment area of the skin of the patient prior to forming any fractional columns in the treatment area. It is also possible for the pharmaceutical composition or drug to be applied to the exterior surface of the skin in a treatment area of the skin of the patient after forming at least one fractional column in the treatment area, or substantially simultaneously with forming at least one fractional column in the treatment area.

Thus, in another aspect, methods of increasing the uptake of a pharmaceutical composition or drug by a patient are described herein. Such a method can comprise applying the pharmaceutical composition or drug to an exterior surface of skin of the patient and forming one or more fractional columns of tissue in the skin of the patient in a manner described above. For example, in some cases, at least one fractional column has a structure along or as a function of a z-direction orthogonal to an exterior surface of the skin, and the structure of the fractional column varies along the z-direction in one or more of angular orientation relative to the exterior surface of the skin, ablated channel width or diameter, coagulation zone thickness, coagulation zone offset in an x-direction or a y-direction perpendicular to the z-direction, coagulation zone intensity, and thermal insult. Additionally, in some embodiments, such a method further comprises determining one or more features of the at least one fractional column based on an amount and/or chemical identity of the pharmaceutical composition. Similarly, in some instances, such a method may also comprise imaging a treatment area of the skin of the patient to obtain an image of the treatment area, in a manner similar to that described above. In some embodiments, for example, the treatment area is imaged before forming one or more fractional columns in the skin of the patient, including within the treatment area. Further, in some cases, a method of increasing the uptake of a pharmaceutical composition or drug described herein comprises diagnosing a condition, disease, malady, disorder, or treatment modality of the skin of the patient based on an image of the treatment area, before forming one or more fractional columns in the skin of the patient. It is further to be understood that one or more features of one or more fractional columns can be determined or selected based on a diagnosed condition in addition to being determined or selected based on the amount or identity of a pharmaceutical composition or drug applied to the patient. Moreover, in some embodiments, the pharmaceutical composition itself is determined or selected based on a diagnosis described hereinabove. Additionally, it is to be understood that a method of increasing the uptake of a pharmaceutical composition by a patient need not be limited to forming only a single fractional column in the skin of the patient. Instead, as described above, a plurality of fractional columns may be formed. Moreover, the plurality of fractional columns can define an array in an xy-plane of the exterior surface of the skin, as described above. The array may or may not be spatially uniform. Additionally, when a plurality of fractional columns is formed, the plurality of fractional columns can be formed in any temporal order described above. Further, each fractional column of the plurality of columns can have a complex structure described above. Moreover, the plurality of columns can have the same complex structure or a plurality of differing complex structures.

In another aspect, systems for the laser treatment of a patient, or of the skin of a patient, are described herein. In some such embodiments, the system comprises a laser configured or adapted to perform, or that performs or carries out, fractional laser ablation. The system also comprises a laser configured or adapted to perform, or that performs or carries out, non-ablative laser coagulation. It is to be understood that the fractional laser ablation and the non-ablative laser coagulation can be of tissue, including living human tissue. In addition, it is further to be understood that, in some cases, a single laser is configured to selectively perform fractional laser ablation and non-ablative laser coagulation. Alternatively, in other instances, the laser configured to perform fractional laser ablation is a first laser and the laser configured to perform non-ablative laser coagulation is a second laser, the first and second lasers differing from one another. Moreover, in some embodiments, whether one laser or more than one laser is used, a plurality of differing ablative wavelengths and/or a plurality of differing non-ablative, coagulative wavelengths may be produced by the one or more lasers of the system and used in a step of a method described herein (where it is to be understood that “differing” ablative or coagulative “wavelengths” refers to doses or exposures of ablative or coagulative laser light, respectively, having differing average wavelengths). For example, as described further below, a system or method described herein can include two differing coagulative laser outputs and two differing ablative laser outputs, or one ablative laser output and three differing coagulative laser outputs, or only one ablative laser output and only one coagulative laser output. Other combinations are also possible.

Systems described herein further comprise a switching component configured to switch output of the system from a fractional laser ablation output to a non-ablative laser coagulation output. Additionally, such systems also comprise a controller configured to direct the system to apply a plurality of doses of fractionally ablative laser light and/or a plurality of doses of non-ablative coagulative laser light to a first spot or location on skin of the patient. In some embodiments, the system applies doses of fractionally ablative laser light and doses of non-ablative coagulative laser light non-simultaneously, such as in an alternating manner. In other cases, the system applies doses of fractionally ablative laser light and doses of non-ablative coagulative laser light simultaneously, such as may be achieved using a plurality of lasers. Moreover, in some instances, a system described herein further comprises one or more lenses, mirrors, and/or actuators for directing the fractional laser ablation output and/or the non-ablative laser coagulation output of the system to one or more desired locations on the skin of the patient.

Further, in some embodiments, a system described herein also comprises an imaging device or system configured to image a treatment area of the skin of the patient. Such an imaging device or system may include computer hardware and/or software for diagnosing a condition, disease, malady, disorder, or treatment modality of the skin of the patient based on the image of the treatment area.

Moreover, in some instances, a system described herein comprises a handpiece having an interior compartment having a proximal end and a distal end, and an optical aperture disposed at the distal end. In such cases, the fractional laser ablation output and/or the non-ablative laser coagulation output of the system can be configured to pass through the interior compartment and out of the optical aperture. In other embodiments, a system described herein comprises an optical fiber having a proximal end and a distal end, and the fractional laser ablation output and/or the non-ablative laser coagulation output of the system is configured to pass through the optical fiber and out of the distal end of the optical fiber.

These and other embodiments are described in more detail in the detailed description which follows.

### BRIEF DESCRIPTION OF THE FIGURES

FIGS. 1A-1C schematically illustrate ablative laser treatment of tissue according to some embodiments described herein.

FIGS. 2A-2C schematically illustrate coagulative laser treatment of tissue according to some embodiments described herein.

FIGS. 3A-3I schematically illustrate fractional columns associated with exemplary combinations of ablative and coagulative laser treatment of tissue according to some embodiments described herein.

FIGS. 4A-4B are graphical illustrations of exemplary ablative and coagulative laser treatment cycles for laser treatment of tissue according to some embodiments described herein.

FIGS. 5A-5J schematically illustrate fractional columns associated with exemplary combinations of ablative and coagulative laser treatment of tissue according to some embodiments described herein.

FIG. 6 is a block diagram of an exemplary system for laser treatment of skin according to some embodiments described herein.

FIGS. 7A-7C schematically illustrate customized arrays for the combined ablative and coagulative laser treatment of tissue according to some embodiments described herein.

### DETAILED DESCRIPTION

Embodiments described herein can be understood more readily by reference to the following detailed description, examples, and figures. Elements, apparatus, and methods described herein, however, are not limited to the specific embodiments presented in the detailed description, examples, and figures. It should be recognized that these embodiments are merely illustrative of the principles of the present invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “from 5 to 10” or “5-10” should generally be considered to include the end points 5 and 10.

Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

### I. Methods of Laser Treatment

In one aspect, methods of laser treatment are described herein, including methods of treating a patient or the skin of a patient, such as a human patient. Such methods, more particularly, can be described as fractional laser treatment methods. As understood by one of ordinary skill in the art, “fractional” laser ablation refers to a laser ablation process in which an ablating laser beam is used to selectively ablate, vaporize, destroy, or remove columns of tissue, or “drill holes,” in a targeted area such as a treatment area of skin. Similarly, “fractional” laser coagulation refers to a laser coagulation process in which a coagulating laser beam is used to selectively coagulate columns or regions of tissue in a targeted area such as a treatment area of skin. More specifically, coagulated tissue can refer to tissue that has been heated (via a coagulative laser) to a temperature or temperature regime that is hot enough to cause coagulation of tissue, but not hot enough to cause ablation of the tissue. For reference purposes herein, such a coagulation temperature regime or zone denotes a tissue temperature of 50-140° C., maintained for a time period insufficient to substantially ablate the tissue. For instance, in some cases, less than 5%, less than 3%, or less than 1% of tissue in or exposed to a coagulation temperature regime described herein is ablated. Further, the tissue temperature of 50-140° C., in some embodiments, is maintained for less than 5 seconds, less than 3 seconds, or less than 1 second.

As described further below, coagulated columns or regions formed by fractional laser coagulation, or columnar vacancies or “holes” formed by fractional laser ablation, can define a pattern or array of columns or vacancies or holes in the targeted area, where the columns or vacancies or holes have a desired diameter, depth, and areal density (of less than 100%) on a treatment area, which may be an exterior surface of skin. Additionally, as described further below, fractional laser ablation or coagulation can be carried out with a variety of spot sizes, scan or exposure patterns, and lasers.

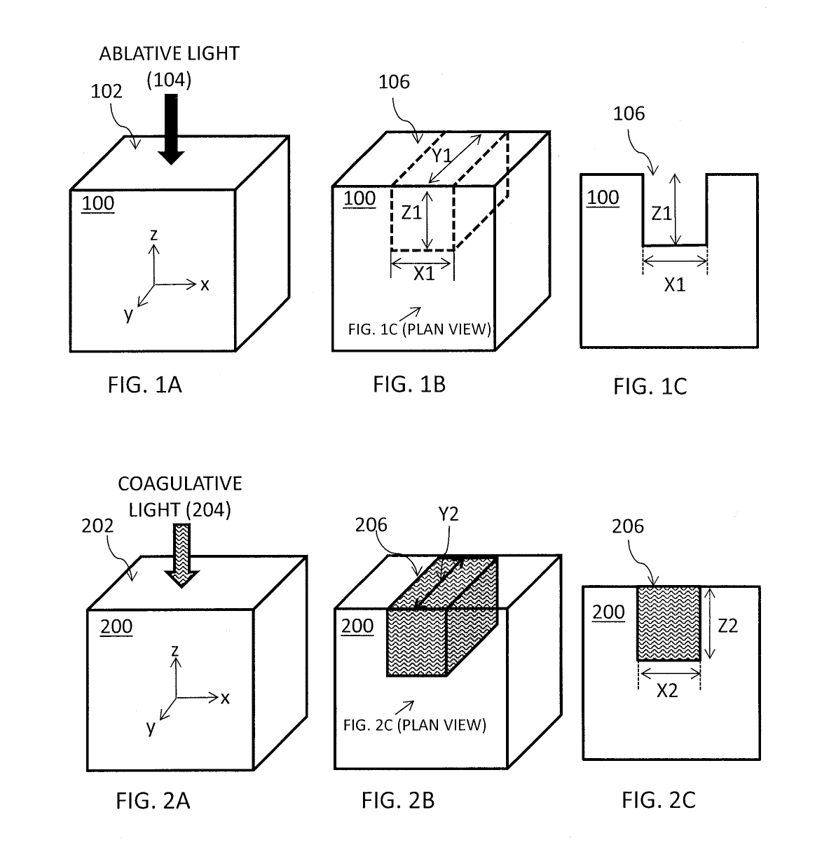

An exemplary fractional laser treatment process is illustrated in FIG. 1 and FIG. 2. Referring now to FIGS. 1A-1C, schematic illustrations of a subject's skin tissue 100 both during and after an ablative laser treatment are shown. As FIG. 1A illustrates, a dose of fractionally ablative laser light 104 is applied to an external surface 102 of the subject's skin during an ablative laser treatment. The laser light 104 may be orthogonally disposed relative the surface 102 of the subject's skin or angled relative the surface 102 of the subject's skin. The laser light 104 may form one or more circular or non-circular ablated columns or channels 106 in the subject's skin.

FIGS. 1B and 1C show respective dimensional and plan views of the skin tissue 100 after application of the ablative laser light 104. In FIG. 1B the ablated channel 106 is shown in broken lines for illustration purposes only, so that the x-, y-, and z-directions are readily visible. As FIGS. 1B and 1C collectively illustrate, each ablated channel 106 that forms during an ablative laser treatment defines a three-dimensional structure having a length Y1, a width X1, and a depth Z1. The length Y1 and/or width X1 of the ablated channel 106 can be symmetric or non-symmetric with respect to the z-axis in the z-direction. For example and in some embodiments, the length Y1 and/or width X1 can vary in the z-direction in a continuous or discontinuous manner. In other embodiments, the length Y1 and/or width X1 do not vary in the z-direction. Each channel 106 can facilitate cutaneous drug delivery. In some embodiments, the uptake (i.e., diffusion) and/or location of the drug delivery can be customized via customizing the size (i.e., diameter, length, width, depth, etc.) and/or shape (i.e., sectional or plan shape) of the ablated channel 106 alone or in combination with alternating (or simultaneous) ablative laser light treatment cycles and non-ablative coagulative laser light treatment cycles.

FIGS. 2A-2C are schematic illustrations of a subject's skin tissue 200 during and after a non-ablative coagulative laser light treatment. As FIG. 2A illustrates, a dose of non-ablative coagulative laser light 204 is applied to a surface 202 of the subject's skin during a non-ablative coagulative laser treatment. The laser light 204 may be orthogonally disposed relative the surface 202 of the subject's skin or angled relative the surface 202 of the subject's skin. The laser light is configured to form one or more coagulation zones 206 in the subject's skin. Coagulation zones 206 include areas (i.e., spots or locations) of coagulated tissue, whereas ablated channels (i.e., 106, FIG. 1C) include areas of eradicated or removed tissue.

As FIGS. 2B and 2C collectively illustrate, each coagulation zone 206 that forms during a coagulative laser treatment is a three-dimensional structure having a length Y2, a width X2, and a depth Z2. The length Y2, width X2, and/or depth Z3 collectively define and/or form a columnar structure that can vary along the z-direction (i.e., the z-axis) in a continuous or discontinuous manner. In some embodiments, the ablative laser light treatment depicted in FIGS. 1A-1C is used in combination with the non-ablative coagulative laser light treatment depicted in FIGS. 2A-2C for providing areas of skin having various structures that facilitate customized cutaneous drug delivery.

As described above, fractional channels, particularly fractional ablation channels, can be used to assist in drug delivery. Previously, such fractional channels for drug delivery have been defined or characterized by three parameters: ablation spot size or width, ablation depth, and the thickness of the coagulation zone that frequently surrounds or envelops or borders the ablated area or channel Moreover, these parameters have previously been uniform or non-varying or substantially non-varying (e.g., varying by less than 5%, less than 3%, or less than 1%) along a depth or z-direction perpendicular to the surface of a treatment area, such as an exterior surface of skin. However, as disclosed herein, some such monolithic fractional channels can provide only limited therapeutic benefit. For instance, such fractional channels can provide only a “single lever” approach to controlling diffusion, bleeding, and oozing in a fractional treatment site, namely, by positively affecting the rate of diffusion of therapeutic molecules into viable tissue and negatively impacting the opposing forces brought upon by the onset of the bleeding and oozing that occurs post-ablation, using the single parameter of coagulation zone thickness, for instance. As described further herein, some methods according to the present disclosure can provide complex fractional channels or columns permitting a “multi lever” approach to diffusion management and other treatment modalities.

In some embodiments, a method described herein comprises forming at least one fractional column or region of skin tissue in a patient, wherein the fractional column has a structure along a z-direction orthogonal to an exterior surface of the skin, and wherein the structure of the fractional column varies along the z-direction in one or more ways. For instance, the structure of the fractional column can vary along the z-direction in one or more of the following ways: angular orientation relative to the exterior surface of the skin; ablated channel width or diameter; coagulation zone thickness; coagulation zone offset in an x-direction or a y-direction perpendicular to the z-direction; coagulation zone intensity; and thermal insult.

For reference purposes herein, it is to be understood that the “angular orientation relative to the exterior surface of the skin” refers to the angle between a vector parallel to the exterior surface of the skin (within a region and at a scale in which the exterior surface of the skin is planar) and a vector corresponding to the long direction of a fractional column of tissue, where the column could comprise an “empty” channel defined by the absence of previously ablated tissue, optionally surrounded by an envelope of coagulated tissue. Thus, if a fractional column (or segment thereof) is formed or oriented “straight down” from the surface, the angular orientation would be 90 degrees.

Similarly, the “ablated channel width or diameter” refers to the width or diameter of the column or channel of tissue removed by ablation at a given depth (i.e., value along the z-direction).

The “coagulation zone thickness” refers to the thickness of coagulated tissue, in a direction perpendicular to the angle of incidence of the laser or perpendicular to the z-direction. It is to be understood, however, that a coagulation zone can exist on more than two sides of an ablated channel. Specifically, a coagulation zone can exist on both sides (perpendicular to the z-direction) and “below” the ablated channel (parallel to the z-direction). However, this “bottom” portion of the coagulation zone can be described herein as another segment (lower down in the z-direction) of the structure of the overall fractional channel.

“Coagulation zone offset in an x-direction” and “coagulation zone offset in a y-direction” perpendicular to the z-direction refer to the axial or concentric symmetry or asymmetry of an ablated tissue column or channel within a coagulated tissue column or region.

The “coagulation zone intensity,” as opposed to the thickness of the coagulation zone, is a measure of the degree of thermal damage of tissue within the coagulation zone. As understood by one of ordinary skill in the art, refers to “coagulation” can range from relatively mild heating/thermal damage to relatively severe heat shock/thermal damage (as measured, for instance, based on the presence or absence of specific protein cascades and/or the physical structure of collagen within the coagulation zone). As described further below, the coagulation zone intensity can be controlled by controlling the thermal density provided by the coagulative laser beam in the middle of the coagulation zone. Additionally, it has been discovered that the coagulation zone intensity can be used to control the thermal diffusion constant of the coagulated tissue.

“Thermal insult” refers to the spatial distribution and total amount of thermal energy present within a 1 cm radius around the center of a fractional column.

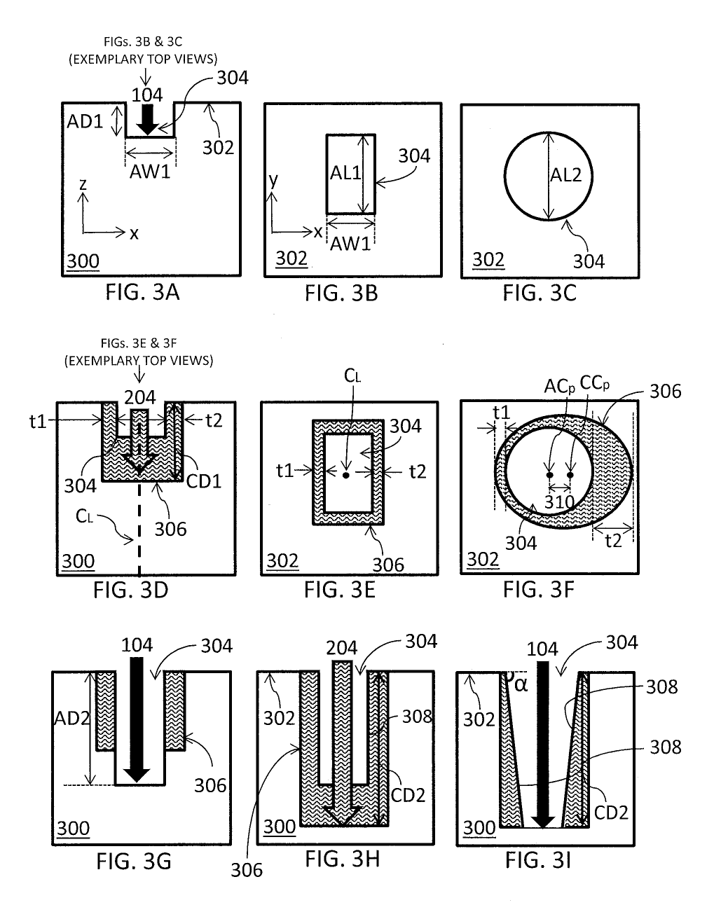

The foregoing parameters can be understood more readily by reference to FIG. 3. FIGS. 3A-3I schematically illustrate various aspects associated with fractional columns that form in a subject's skin 300 during one or more cycles of ablative and coagulative laser light treatments according to some embodiments described herein. In FIGS. 3A-3I, each drawing that shows a plan view of the skin 300 is taken along the y-direction, and each drawing that shows a plan view of a surface 302 of the skin 300 is taken along the z-direction as indicated by the axes in FIGS. 3A and 3B. For illustration purposes only, the axes are not repeated in each drawing.

FIG. 3A is a sectional view of a subject's skin 300 and a channel 304 that forms in a surface 302 thereof during treatment with an ablative laser light 104. The channel 304 can have an ablative depth AD1 in the z-direction, an ablative width AW1 in the x-direction, and an ablative length AL1 in the y-direction. As FIG. 3B illustrates, the channel 304 may form a non-circular structure having a non-circular surface area. Alternatively and as FIG. 3C illustrates, the channel 304 may be a circular structure having a circular surface area and an ablative diameter AL2 in the y-direction. The fractional column structures that form as a result of alternating ablative and non-ablative laser treatments as described herein may include substantially cubical columns, cylindrical columns, conical columns, or non-cylindrical columns that have regular or irregularly shaped sections. Where a plurality of columns are formed in a subject's skin, the elongated axis (i.e., along the z-axis) of each column may be substantially parallel or non-parallel, where desired.

As FIG. 3B illustrates, a dose of non-ablative coagulative laser light 204 can be applied to a subject's skin after formation of the ablative channel 304. The dose of non-ablative coagulative laser light 204 can form a coagulation zone 306 around portions of the ablative channel 304. For example, the coagulation zone can form over, on, and/or around the ablative depth, width, and length (i.e., AD1, AW1, AL1, etc.) of the ablative channel 304. The coagulation zone 306 can extend along the z-axis from the surface 302 to points below a floor of the channel 304, and have an overall coagulation depth CD1 in the z-direction. The coagulation zone 306 is also wider than the ablative channel, and may include a first thickness t1 in the x-direction on a first side of the channel 304 and a second thickness t2 in the x-direction on a second, opposing side of the channel 304.

In some embodiments as illustrated in FIG. 3E, the respective channel 304 and coagulation zone 306 can be concentric and/or coaxially aligned along a centerline C_{L} of the channel 304. The channel 304 and coagulation zone 306 can be concentric and symmetrically disposed with respect to the centerline C_{L} of the channel 304, or the channel 304 and coagulation zone 306 can be concentric and asymmetrically disposed with respect to the centerline C_{L}. That is, one of the channel 304 and the coagulation zone 306 may be asymmetric with respect to the centerline C_{L} but still remain concentric to the other structure.

In further embodiments as illustrated in FIG. 3F, the channel 304 and coagulation zone 306 are not concentric. For example, the ablation channel 304 has a respective center point AC_{P} and the coagulation zone 306 has a respective center point CC_{P}. The different center points can be spaced apart or offset by a given distance 310. The coagulation zone 306 can be offset in the x-direction, the y-direction, or perpendicular to the z-direction. The thickness t1 of coagulation zone 306 on one side of the channel 304 can be greater than the opposing thickness t2 of coagulation zone 306 on the opposing side of the channel 304.

FIGS. 3G-3I illustrate further aspects associated with fractional laser treatments and the resulting structures in a subject's skin 300. As FIG. 3G illustrates, a subsequent dose of ablative laser light 104 can be applied to the subject's skin 300 after the initial dose that is applied in FIG. 3A. The subsequent dose of ablative laser light 104 can be applied after formation of coagulation zone 306. Multiple cycles of ablative laser light 104 and non-ablative coagulative light 204 can be alternated and applied to the skin 300 for treating various skin conditions, including treatment of skin conditions via cutaneous drug delivery. The length, width, and/or depth of columnar structures comprised of one or more channels 304 combined with one or more coagulation zones 306 can be customized according to the uptake of a given pharmaceutical composition to be delivered or other aspect associated with drug delivery, such as a subject's gender or weight. After multiple ablative laser treatments, the channel 304 includes a greater ablative depth AD2 in the z-direction.

FIGS. 3H and 3I illustrate aspects associated with subsequent doses of non-ablative coagulative light 204 being applied to a subject's skin 300 after the initial dose in FIG. 3D. The subsequent doses of non-ablative coagulative light 204 can increase the length, width, diameter, and/or depth of the coagulation zone 306. For example, after application of the subsequent dose of non-ablative coagulative light 204 the depth of the coagulation zone 306 can increase to CD2 in the z-direction. As FIG. 3H illustrates, channel 304 can comprise an inner wall 308 and/or walls that are substantially parallel to each other. The inner walls 308 can also be substantially orthogonal to the surface 302 of skin 300. In other embodiments as illustrated in FIG. 3I, the inner wall 308 and/or walls are not substantially parallel to each other and not substantially orthogonal to the surface 302 of skin 300. For example, the inner wall(s) 308 can be disposed at an angle α (alpha) relative to the surface 302 of skin 300.

Turning again to the structure of fractional columns according to the present disclosure, in some cases, the structure of the fractional column along the z-direction is defined by a plurality of segments that differ in one or more ways. For example, in some embodiments, the segments differ in one or more of the structural parameters identified above, such as angular orientation relative to the exterior surface of the skin, ablated channel width, coagulation zone thickness, coagulation zone offset in an x-direction or a y-direction perpendicular to the z-direction, coagulation zone intensity, and thermal insult. More particularly, in some instances, the plurality of segments differ in angular orientation relative to the exterior surface of the skin. In other cases, the plurality of segments differ in ablated channel width and/or coagulation zone thickness. The plurality of segments may also differ in coagulation zone offset in the x-direction or the y-direction, and/or in coagulation zone intensity. Moreover, in some embodiments described herein, the plurality of segments differ in thermal insult.

Additionally, as described above, the plurality of segments can vary in a continuous or discontinuous manner. In some cases, for example, the plurality of segments define a step function with respect to one or more of the angular orientation relative to the exterior surface of the skin, ablated channel width, coagulation zone thickness, coagulation zone offset in an x-direction or a y-direction perpendicular to the z-direction, coagulation zone intensity, and thermal insult. Alternatively, in other instances, the plurality of segments define a continuous function with respect to one or more of the angular orientation relative to the exterior surface of the skin, ablated channel width, coagulation zone thickness, coagulation zone offset in an x-direction or a y-direction perpendicular to the z-direction, coagulation zone intensity, and thermal insult. Structures such as those described above can exhibit complex molecular (e.g., drug) diffusion profiles and/or thermal insult profiles. In some cases, for example, at least two segments of the plurality of segments have different molecular diffusion rates and/or different temperature gradients in a lateral direction orthogonal to the z-direction. It is to be understood that such a temperature gradient is spatial temperature gradient at a given time point (such as 1 ms, 5 ms, or 10 ms after formation of the segments), since temperatures in a given spatial region including thermally conductive material will change over time due to thermal transport.

Specific steps of methods described herein will now be further described in more detail. Methods described herein comprise forming one or more fractional columns or regions of tissue in skin of a patient. The skin of the patient can be any skin of any patient not inconsistent with the objectives of the present disclosure. In some embodiments, the skin is exterior or surface skin of a human patient. Further, in some instances, the patient being treated, or whose skin is being treated, is in need of the treatment, including due to the presence of a condition, disease, malady, or disorder, of the skin or otherwise.

Additionally, the one or more fractional columns or regions of tissue can be formed in the skin of the patient in any manner not consistent with the objectives of the present disclosure. For example, in some embodiments, forming the at least one fractional column comprises, or is carried out by, applying a plurality of doses of fractionally ablative laser light and/or one or more doses of non-ablative coagulative laser light to the skin of the patient. In some cases, forming at least one fractional column comprises or is carried out by applying a plurality of doses of fractionally ablative laser light and a plurality of doses of non-ablative coagulative laser light to a first spot or location on the skin of the patient, thereby forming the fractional column having a complex structure. Further, the plurality of doses can be applied in a simultaneous or non-simultaneous manner. For instance, in some cases, the plurality of doses of ablative laser light and the plurality of doses of coagulative laser light are applied in a sequential or alternating manner. In other embodiments, one or more doses of ablative laser light and one or more doses of coagulative laser light are applied simultaneously, such as may be achieved using a plurality of lasers.

It is to be understood, for reference purposes herein, that a “dose” (or “exposure”) of laser light is generally not synonymous with a “pulse” of laser light, particularly not with respect to the “pulses” of laser light inherently produced by a pulsed laser (as opposed to a continuous wave laser). Instead, a “dose” of laser light in the context of the present disclosure refers to light emitted by a laser during a single, discrete “on” time of the laser, during which the laser light is directed to a treatment area described herein (or to a single spot or location within the treatment area). Moreover, the “dose” of laser light can have a duration that is greater than the pulse duration of a pulsed laser (if a pulsed laser is used). For example, in some cases, a single “dose” of laser light is at least 1 ms, at least 5 ms, at least 10 ms, at least 100 ms, at least 0.5 seconds, or at least 1 second in duration. In some cases, a “dose” of laser light described herein has a duration of 1 ms to 10 seconds, 1 ms to 5 seconds, 1 ms to 1 second, 100 ms to 10 seconds, 100 ms to 5 seconds, or 100 ms to 1 second. Moreover, a “dose” of laser light is temporally bounded on both sides by an “off” period of time during which the laser light is not directed to or incident on the treatment area (or on the single spot or location within the treatment area). Further, this “off” period of time is longer than (and different from) the time between pulses generated by a pulsed laser in continuous operation (if a pulsed laser is used).

Similarly, as understood by one of ordinary skill in the art, “ablative laser light” or an “ablative laser beam” refers to laser light or a laser beam of sufficient peak power and irradiation duration to ablate, vaporize, destroy, and/or remove biological tissue irradiated by the laser light or laser beam. Similarly, “coagulative laser light” or a “coagulative laser beam” refers to laser light or a laser beam of sufficient peak power and irradiation duration to heat irradiated biological tissue to a temperature within a coagulation regime described herein, but not of sufficient peak power and irradiation duration to ablate or substantially ablate the irradiated tissue (where “substantial ablation” refers to ablation of 1% or more, 3% or more, or 5% or more of irradiated tissue, as described above). Thus, “ablative laser light” (or an “ablative laser beam”) and “coagulative laser light” (or a “coagulative laser beam”) are mutually exclusive terms as used herein. That is, laser light (or a laser beam) that is “ablative” for irradiated tissue in a given instance is not also “coagulative” for the irradiated tissue in that same instance, except as may occur incidentally: as understood by one of ordinary skill in the art, ablative laser light (or an ablative laser beam), when used fractionally, can also cause a relatively small amount of coagulation on the sidewalls of ablated channels or “holes” formed by the ablative laser light (or beam). It is to be understood that such “incidental” coagulation (which may represent less than 10%, less than 5%, less than 3%, or less than 1% of the total mass of tissue affected (i.e., primarily ablated) by the ablative laser light) does not mean that laser light (or a laser beam) that is otherwise ablative is also “coagulative” as the terms “ablative” and “coagulative” are used herein to modify the terms “laser light,” “laser,” or “laser beam.”

The depth of ablation in an ablation step can vary. Any depth not inconsistent with the objectives of the present disclosure may be used. For example, in some embodiments, an ablation step removes at least 90%, at least 95%, at least 98%, or at least 99% of tissue in a column of a given width to a depth of up to 1000 μm or to a depth of up to 2000 μm. In some cases, an ablation step removes at least 90%, at least 95%, at least 98%, or at least 99% of tissue in the column to a depth of 50-2000 μm, 50-1000 μm , 50-500 μm, 50-300 μm, 50-200 μm, 100-2000 μm, 100-1000 μm, 100-500 μm, 100-300 μm, 100-200 μm, 200-2000 μm, 200-1000 μm, 200-500 μm, 400-2000 μm, 400-1000 μm, 500-2000 μm, 500-1000 μm, or 1000-2000 μm.

The depth and areal density of ablation in a fractional laser ablation step described herein can vary. Any depth and areal density not inconsistent with the objectives of the present disclosure may be used. For example, in some preferred embodiments, the fractional laser ablation generates holes in up to 25% or up to 35% of the surface area of the treatment area, the holes having an average diameter of 150-600 μm and an average depth of up to 2 mm. In other cases, the fractional laser ablation generates holes in 15-35%, 15-30%, 15-25%, 20-35%, or 20-30% of the surface area of the treatment area, wherein the holes have an average diameter of 150-500 μm, 150-450 μm, 150-400 μm, 200-600 μm, 200-500 μm, 200-450 μm, 200-400 μm, 250-600 μm, 250-500 μm, 250-450 μm, 250-400 μm, 300-600 μm, 300-500 μm, 300-450 μm, 300-400 μm, 400-600 μm, 400-500 μm, or 450-600 μm, and a depth of 0.3-2.5 mm, 0.3-2 mm, 0.3-1.5 mm, 0.3-1 mm, 0.5-2.5 mm, 0.5-2 mm, 0.5-1.5 mm, 0.5-1 mm, 1-2.5 mm, or 1-2 mm.

A laser, laser light, or a laser beam of a method described herein can have any power and any peak or average emission wavelength not inconsistent with the objectives of the present disclosure, provided that, in a given instance, the laser light or laser beam characteristics correspond to the desired effect (e.g., ablation or coagulation). For example, in some embodiments, a laser or laser beam of a device described herein has a peak or average emission wavelength in the infrared (IR) region of the electromagnetic spectrum. In some such cases, the laser or laser beam has a peak or average emission wavelength in the range of 1-4 μm, 1-3 μm, 2-4 μm, 2-3 μm, 8-12 μm, or 9-11 μm. For example, in some embodiments, the laser or laser beam comprises an erbium-doped yttrium aluminum garnet (Er:YAG) laser or laser beam or a neodymium-doped YAG (Nd:YAG) laser or laser beam having a peak or average emission wavelength of 2940 nm or 1064 nm. In other cases, the laser or laser beam comprises a carbon dioxide laser or laser beam. A laser beam described herein can also have a peak or average emission wavelength in the visible region of the electromagnetic spectrum. Non-limiting examples of peak or average emission wavelengths suitable for use in some embodiments described herein include 532 nm, 695 nm, 755 nm, 1064 nm, and 1470 nm (e.g., for non-ablative application), or 2940 nm (e.g., for ablative application). Further, in some instances, a laser or laser beam of a device described herein has an average power of 1 to 100 W (e.g., when used for coagulation) or 5 to 200 W (e.g., when used for ablation). Additionally, it is to be understood that a “laser” can refer to a single lasing device that produces a single beam of laser light from a single lasing medium at a time. However, in some embodiments, the laser comprises a hybrid laser operable to produce laser beams having a plurality of differing wavelengths. For instance, in some cases, the hybrid laser is operable to selectively produce an ablative laser beam and a coagulative laser beam. Additionally, in some cases, one or more lasers used in a method or system described herein can selectively produce a plurality of differing ablative laser outputs (e.g., having differing ablative wavelengths) and/or a plurality of differing coagulative laser outputs (e.g., having differing non-ablative wavelengths). Moreover, in some such instances, any combination of ablative and non-ablative lasers or laser beams may be used. For example, in a single method described herein, one ablative laser/laser beam and two differing coagulative lasers/laser beams could be used. Other combinations are also possible.

Moreover, the spot size of a laser beam produced by a laser described herein may also vary. Any spot size not inconsistent with the objectives of the present disclosure may be used. In some cases, for instance, the spot size is 0.1-10 mm, 0.1-1 mm, 0.1-0.5 mm, 0.5-5 mm, 1-10 mm, or 1-5 mm. Other spot sizes may also be used.

A laser of a method or system described herein may also be a pulsed laser or a continuous wave (CW) laser. Moreover, when a pulsed laser is used, the laser can produce time-modulated pulses of the laser beam. For instance, in some cases, the laser beam comprises an ablative laser beam and the laser produces time-modulated pulses of the ablative laser beam. Not intending to be bound by theory, it is believed that the use of such a pulsed laser beam can provide both ablation and coagulation. More particularly, in some embodiments, time-modulated pulses of an ablative laser beam produce tissue ablation in an ablation area, followed by tissue coagulation around the ablation area.

Additionally, in some preferred embodiments, a laser ablation or coagulation step is carried out using a laser scanner. A “laser scanner,” for reference purposes herein, refers to an apparatus which can be attached to a laser system for delivery of a laser beam over an area defined by the operator and assisted by a computer control system which is larger than a single spot of the laser beam. A typical construction of this apparatus involves an opto-mechanical arrangement of two orthogonal motors with mirrors mounted on them which receive the laser beam and are controlled by a computer control system. Each motor or actuator is capable of directing the beam in an axis. The combination of two orthogonal motors/mirrors allows the scanner to draw any arbitrary pattern in two dimensions (e.g., x and y) on the tissue or other targeted area.

Moreover, with reference once again to the variable structural parameters of fractional columns described hereinabove, a method described herein can comprise tuning or varying one or more of these structural parameters in accordance with Table I below, including while forming a fractional column in a manner described hereinabove. For example, in some cases, the angular orientation of a fractional column (or of a segment of a fractional column) relative to the exterior surface of the skin is selected by altering the angle of incidence of a laser, laser light, or laser beam described herein, including using optical and/or mechanical means or components, such as one or more lenses, mirrors, and/or actuators.

TABLE I
Methods of Varying Structural Parameters of a Fractional Column.

| Structural Parameter | |
|---|---|
| of Fractional Column | Method of Selecting Value of Structural |
| (or Segment Thereof) | Parameter |
| angular orientation | selecting angle of incidence of ablative and/or |
| relative to the exterior | coagulative laser beam using optical and/or |
| surface of the skin | mechanical components of laser system |
| ablated channel depth | selecting one or more of: duration of ablative |
| | laser exposure or dose, average or peak |
| | wavelength of ablative laser, and laser energy |
| | density |
| ablated channel width | selecting one or more of: duration of ablative |
| | laser exposure or dose, average or peak |
| | wavelength of ablative laser, laser energy |
| | density, spot size of laser, or raster scanning of |
| | a laser spot opto-mechanically |
| coagulation zone | selecting one or more of: duration of |
| thickness | coagulative laser exposure or dose, f-number |
| | of coagulative laser beam, spot size/diameter |
| | of coagulative laser beam, average or peak |
| | wavelength of coagulative laser beam, and |
| | cumulative effect of sequential coagulative |
| | laser exposures or doses |
| coagulation zone offset | selecting the axial or concentric symmetry or |
| in an x-direction or a y- | asymmetry of an ablated tissue column or |
| direction perpendicular | channel within a coagulated tissue column or |
| to the z-direction | region, generally opto-mechanically |
| coagulation zone | selecting the thermal density provided by the |
| intensity | incident laser beam in the middle of the |
| | coagulation zone, which can be done by |
| | selecting one or more of the beam shape, the |
| | beam diameter, the beam power, the beam |
| | peak power, the beam peak or average |
| | wavelength, and the beam exposure time or |
| | dose |
| thermal insult | selecting the coagulation zone thickness and/or |
| | the thermal density provided by the incident |
| | laser beam in the middle of the coagulation |
| | zone (e.g., by selecting the peak or average |
| | wavelength of the incident laser beam) |

In addition to the structural parameters identified above, one or more fractional columns (or one or more segments of a given fractional column) can be further defined, in the context of a method described herein, by one or more of the following structural or temporal properties: x- and/or y-position of the column (or segment thereof) within a treatment area (where the surface of the treatment area or skin is taken to define the xy-plane); and the order of formation of a specific fractional column (or segment thereof) relative to other fractional columns (or segments thereof) in an array of columns (or within the single fractional column). In the case of the x- and/or y-position, this parameter can be selected by directing one or more laser beams to a desired location opto-mechanically, or by selecting a laser beam having a complex beam shape. The order of formation of a fractional column within an array (or of a segment within a single fractional column) can likewise be selected by directing one or more laser beams to a desired location at a desired time opto-mechanically, or by selecting a laser beam having a complex beam shape.

As described above, in some embodiments, a method described herein comprises forming a plurality of fractional columns of tissue in the skin of the patient, wherein one or more of the plurality of fractional columns can have a structure in the z-direction as described hereinabove. Moreover, differing fractional columns of the plurality of fractional columns can have differing structures in the z-direction or the same structure in the z-direction. Further, the plurality of fractional columns can define an array in an xy-plane defined by the exterior surface of the skin of the patient. In general, the fractional columns differ in position on the xy-plane (in terms of having differing x,y coordinates and in terms of having different positions relative to one another). Any array not inconsistent with the objectives of the present disclosure may be used. For example, in some cases, the array is an ordered array of linear “rows and columns” of fractional columns, as defined on the xy-plane. In other instances, the array is a regularly patterned or symmetric array on the xy-plane but does not necessarily include ordered rows and columns of fractional columns In still other embodiments, a plurality of fractional columns forms a non-ordered or random “array” of fractional columns on the xy-plane.

Further, such an array can be formed in any manner not inconsistent with the objectives of the present disclosure. For example, in some cases, the fractional columns are formed substantially simultaneously, such as may occur when a plurality of laser beams are used or when one or more laser beams having a complex beam shape (which may include a plurality of “spots”) is used. In some embodiments, a laser described herein has a beam shape that simultaneously contains the properties necessary (e.g., peak power, number of spots, spot size, spot location) to form a plurality of fractional columns in the same laser firing. In other embodiments, the fractional columns are formed sequentially, such as may occur when a single laser beam is used. In some such cases, a first fractional column is formed at a first location on the xy-plane before a second fractional column is formed at a second location on the xy-plane, the second location differing from the first location.

In addition, it is to be understood that one or more of the plurality of fractional columns can be formed in any manner described herein for forming a single fractional column. Thus, for example, in some embodiments, forming at least one of the plurality of fractional columns comprises, or is carried out by, applying a plurality of doses of fractionally ablative laser light to the skin of the patient and/or applying one or more doses of non-ablative coagulative laser light to the skin of the patient. In some instances, forming at least one of the plurality of fractional columns comprises or is carried out by applying a plurality of doses of fractionally ablative laser light and a plurality of doses of non-ablative coagulative laser light to a first spot or location on the skin of the patient.

Methods described herein, in some embodiments, also comprise imaging a treatment area of the skin of the patient to obtain an image of the treatment area. Such an imaging step can be carried out in any manner and using any imaging device or system not inconsistent with the objectives of the present disclosure. For example, in some embodiments, imaging the treatment area of the skin of the patient is carried out using a technology such as optical coherence tomography (OCT), multi-photon imaging, reflectance confocal microscopy (RCM), fluorescence spectroscopy, camera recognition and image processing, acoustic imaging, or any other imaging technology.

Additionally, in some cases, one or more fractional columns are formed within the treatment area of the skin of the patient, and the treatment area is imaged before forming the one or more fractional columns in the skin of the patient. Imaging prior to forming fractional columns, in some cases, can permit the structure of one or more of the fractional columns, or the order of formation of fractional columns in an array, to be selected based on information provided by the imaging step, including information related to a diagnosis of the skin or of the patient.

For instance, in some embodiments, a method described herein further comprises diagnosing a condition, disease, malady, or disorder of the skin of the patient based on the image of the treatment area, before forming the at least one fractional column in the skin of the patient. Moreover, in some such cases, an image or other information obtained from the imaging step is displayed, processed, or analyzed, including by a computer comprising appropriate hardware and/or software. The display, processing, or analysis of such information can be used to diagnose the skin condition, including in an automated manner. The diagnosis, in turn, can be used, in some cases, to determine or select one or more features of one or more fractional columns, including for purposes of improving efficacy of subsequent treatment of the diagnosed condition, including by application of a drug or pharmaceutical composition. Thus, in some cases, a method described herein further comprises determining one or more features of at least one fractional column based on the diagnosed condition of the skin, before forming the at least one fractional column in the skin of the patient. Similarly, in some instances, a method described herein comprises determining an order of forming a plurality of fractional columns based on the diagnosed condition of the skin, before forming any fractional columns in the skin of the patient.

Methods described herein, in some embodiments, also comprise applying a drug or pharmaceutical composition to the exterior surface of the skin. Any drug or pharmaceutical composition not inconsistent with the objectives of the present disclosure may be used. Some exemplary drugs or pharmaceutical compositions are described further hereinbelow in the specific examples. Additionally, a drug or pharmaceutical composition can be applied to a patient in any manner not inconsistent with the objectives of the present disclosure. For example, in some cases, a pharmaceutical composition is applied to the exterior surface of the skin in a treatment area of the skin of the patient prior to forming any fractional columns in the treatment area. Alternatively, in other instances, a pharmaceutical composition is applied to the exterior surface of the skin in a treatment area of the skin of the patient after forming at least one fractional column in the treatment area. It is also possible to apply a pharmaceutical composition to the exterior surface of the skin in a treatment area of the skin of the patient substantially simultaneously with forming at least one fractional column in the treatment area.

Various aspects of methods of laser treatment have been described hereinabove. It is to be particularly understood that a method described herein can include any combination of steps or features described hereinabove not inconsistent with the objectives of the present disclosure.

### II. Methods of Increasing the Uptake of a Pharmaceutical Composition

In another aspect, methods of increasing the uptake of a pharmaceutical composition are described herein. Such a method can comprise carrying out a method of laser treatment described hereinabove in Section I. Any method of laser treatment described hereinabove in Section I may be used as part of a method of increasing the uptake of a pharmaceutical composition. In some cases, for instance, a method of increasing the uptake of a pharmaceutical composition by a patient comprises applying the pharmaceutical composition to an exterior surface of skin of the patient and forming at least one fractional column or region of tissue in the skin of the patient, wherein the fractional column has a structure along a z-direction orthogonal to an exterior surface of the skin, and wherein the structure of the fractional column varies along the z-direction in one or more of ways described in Section I. For example, in some cases, the structure of the fractional column varies along the z-direction in one or more of angular orientation relative to the exterior surface of the skin, ablated channel width or diameter, coagulation zone thickness, coagulation zone offset in an x-direction or a y-direction perpendicular to the z-direction, coagulation zone intensity, and thermal insult.

Additionally, a method described herein, in some instances, further comprises determining one or more features of the at least one fractional column based on an amount and/or chemical identity of the pharmaceutical composition. For example, in some cases, a fractional column has a coagulation zone thickness that decreases, in a continuous or stepwise fashion, as a function of increasing depth along the z-axis beneath the surface of the skin. Such a structure may be particularly selected or determined based on the use of a pharmaceutical composition or drug having a relatively low molecular diffusion rate in the biological tissue and/or for a pharmaceutical composition or drug used in a small amount. The use of such a structure can permit or facilitate the diffusion of the pharmaceutical composition out of the fractional column and into surround tissue of the patient in increasing amounts and/or at an increasing rate as a function of increasing depth. In this manner, the efficiency of treatment using the pharmaceutical composition or drug can be increased. Additional, non-limiting instances are provided hereinbelow in the specific examples.

A method described herein, in some embodiments, also comprises imaging a treatment area of the skin of the patient to obtain an image of the treatment area. Such an imaging step can be carried out using any imaging system or device and/or in any other manner not inconsistent with the objectives of the present disclosure. In particular, any imaging device or system or other manner of carrying out an imaging step described hereinabove in Section I may be used. In some cases, for example, forming one or more fractional columns in the skin of the patient comprises forming at least one fractional column within the treatment area of the skin of the patient, and imaging the treatment area is carried out before forming the at least one fractional column in the skin of the patient. Similarly, in some instances, imaging the treatment area of the skin of the patient is carried out using OCT, multi-photon imaging, RCM, fluorescence spectroscopy, camera recognition and image processing, acoustic imaging, or any other imaging technology.

Moreover, in some embodiments, a method of increasing the uptake of a pharmaceutical composition or drug described herein comprises diagnosing a condition (or disease, malady, disorder, or treatment modality) of the skin of the patient based on an image of the treatment area, or based on other information obtained from the image. In addition, in some cases, such a diagnosis is carried out before forming a fractional column in the skin of the patient. Such a diagnosing step can be carried out in any manner not inconsistent with the objectives of the present disclosure, including in a manner described hereinabove in Section I.

It is further to be understood that one or more features of one or more fractional columns can be determined or selected based on a diagnosed condition in addition to being determined or selected based on the amount or identify of a pharmaceutical composition applied to the patient. Moreover, in some embodiments, the pharmaceutical composition itself is determined or selected based on a diagnosis described hereinabove.

It is also to be understood that a method of increasing the uptake of a pharmaceutical composition by a patient need not be limited to forming only a single fractional column in the skin of the patient. Instead, as described in Section I above, a plurality of fractional columns may be formed. Moreover, the plurality of fractional columns can define an array in an xy-plane of the exterior surface of the skin, as described above. Additionally, when a plurality of fractional columns are formed, the plurality of fractional columns can be formed in any order described hereinabove in Section I. Further, each fractional column of the plurality of columns can have a complex structure described above in Section I. Moreover, the plurality of columns can have the same complex structure or a plurality of differing complex structures.

### III. Systems for Laser Treatment

In still another aspect, systems for laser treatment of a patient (and/or for increasing the uptake of a pharmaceutical composition by a patient) are described herein. Such a system, in some cases, can be used to carry out a method described hereinabove in Section I or Section II.

In some embodiments, a system described herein comprises a laser configured or adapted to perform, or that performs or carries out, fractional laser ablation. The system also comprises a laser configured or adapted to perform, or that performs or carries out, non-ablative laser coagulation. It is to be understood that the fractional laser ablation and the non-ablative laser coagulation can be of tissue, including living human tissue. In addition, it is further to be understood that, in some cases, a single laser is configured to selectively perform fractional laser ablation and non-ablative laser coagulation. Alternatively, in other instances, the laser configured to perform fractional laser ablation is a first laser and the laser configured to perform non-ablative laser coagulation is a second laser, the first and second lasers differing from one another.

Systems described herein further comprise a switching component configured to switch output of the system from a fractional laser ablation output to a non-ablative laser coagulation output. Additionally, such systems also comprise a controller configured to direct the system to apply a plurality of doses of fractionally ablative laser light and a plurality of doses of non-ablative coagulative laser light to a first spot or location on skin of the patient. In some embodiments, the system applies doses of fractionally ablative laser light and doses of non-ablative coagulative laser light non-simultaneously, such as in an alternating manner. In other cases, the system applies doses of fractionally ablative laser light and doses of non-ablative coagulative laser light simultaneously, such as may be achieved using a plurality of lasers. Moreover, in some instances, a system described herein further comprises one or more lenses, mirrors, and/or actuators for directing the fractional laser ablation output and/or the non-ablative laser coagulation output of the system to one or more desired locations on the skin of the patient.

Further, in some embodiments, a system described herein also comprises an imaging device configured to image a treatment area of the skin of the patient. Such an imaging system may include computer hardware and/or software for diagnosing a condition, disease, malady, disorder, or treatment modality of the skin of the patient based on the image of the treatment area.

Moreover, in some instances, a system described herein comprises a handpiece having an interior compartment having a proximal end and a distal end, and an optical aperture disposed at the distal end. In such cases, the fractional laser ablation output and/or the non-ablative laser coagulation output of the system can be configured to pass through the interior compartment and out of the optical aperture. In other embodiments, a system described herein comprises an optical fiber having a proximal end and a distal end, and the fractional laser ablation output and/or the non-ablative laser coagulation output of the system is configured to pass through the optical fiber and out of the distal end of the optical fiber.

Attention will now be turned once again, in more detail, to specific components of systems described herein. Systems described herein comprise one or more lasers, wherein the one or more lasers are configured or adapted to carry out fractional laser ablation and non-ablative laser coagulation. Any laser, or set of lasers, not inconsistent with the objectives of the present disclosure may be used in a system described herein. In some cases, the laser or set of lasers comprises one or more lasers described hereinabove in Section I. For example, in some instances, one or more lasers of a system described herein comprises has a peak or average emission wavelength in the infrared (IR) region of the electromagnetic spectrum. In some such cases, the laser has a peak or average emission wavelength in the range of 1-4 μm, 1-3 μm, 2-4 μm, 2-3 μm, 8-12 μm, or 9-11 μm. For example, in some embodiments, the laser is an Er:YAG laser or an Nd:YAG laser having a peak or average emission wavelength of 2940 nm or 1064 nm. In other cases, the laser comprises or is a carbon dioxide laser. A laser of a system described herein can also have a peak or average emission wavelength in the visible region of the electromagnetic spectrum and/or an average power described hereinabove in Section I. Similarly, in some embodiments, a laser of a system described herein has a spot size described hereinabove in Section I, such as a spot size of 0.1-10 mm. A laser of a system described herein may also be a pulsed laser or a continuous wave (CW) laser.

A system described herein also comprises a switching component configured to switch output of the system from a fractional laser ablation output to a non-ablative laser coagulation output. Any switching component not inconsistent with the objectives of the present disclosure may be used. In some embodiments, for example, the switching component includes hardware and/or software configured to switch output of the system from a fractional laser ablation output mode to a non-ablative laser coagulation output mode. In some cases, the switching component comprises or is a special purpose computer configured to improve the technological field of laser therapy treatments. More particularly, the switching component of a system described herein can be configured to receive, as input, current laser output data and construct a digitized model or map of a fractional column based on the input. For example, a switching component can comprise a processor configured to execute a biological tissue ablation and/or coagulation simulation module stored in the memory of the switching component. The ablation and/or coagulation simulation module is configured to input laser output data and possibly biological tissue data and construct a digitized model or map of the effect of the laser output on the biological tissue. The simulated effect on the biological tissue can then be used by the switching component to switch the system between ablative and non-ablative laser output modes. A switching component of a system described herein can also comprise one or more opto-mechanical components, such as one or more actuators, lenses, or mirrors. As understood by one of ordinary skill in the art, these components can be used to switch the laser output of the system.

Additionally, systems described herein further comprise a controller configured to direct the system to apply one or more doses of fractionally ablative laser light and/or one or more doses of non-ablative coagulative laser light to a one or more spots or locations on skin of a patient. Any controller not inconsistent with the objectives of the present disclosure may be used. In some cases, the controller is a special purpose computer configured to improve the technological field of laser therapy treatments. More particularly, the controller of a system described herein can be configured to receive, as input, imaging data and construct a digitized map of a treatment area of skin. For example, the controller can comprise a processor configured to execute a feature recognition module stored in the memory of the controller. The feature recognition module can be configured to input imaging data and construct a digitized map of specific locations on the skin for placing one or more laser spots, relative to the total treatment area. The controller can output (e.g., via a wired or wireless connection) control signals or commands instructing components of the system (e.g., one or more actuators, mirrors, or lenses) to move a laser output beam of the system to positions or locations relative to the patient's skin that are centered over one of the desired coordinates corresponding to a target locations. Once the system is in a desired position or configuration, the controller can output control signals or commands instructing the laser to generate an ablative and/or coagulative laser beam.

It is to be understood that a switching component and/or a controller of a system described herein can be implemented in software in combination with hardware and/or firmware. For example, the subject matter described herein can be implemented in software executed by a processor. As used herein, the terms “function” and “module” refer to hardware, firmware, or software in combination with hardware and/or firmware for implementing features described herein. In an exemplary implementation, the subject matter described herein can be implemented using a non-transitory computer readable medium having stored thereon computer executable instructions that when executed by the processor of a computer control the computer to perform steps. Exemplary computer readable media suitable for implementing the subject matter described herein include non-transitory computer-readable media, such as disk memory devices, chip memory devices, programmable logic devices, and application specific integrated circuits. In addition, a computer readable medium that implements the subject matter described herein may be located on a single device or computing platform or may be distributed across multiple devices or computing platforms.

Moreover, in some instances, a system described herein further comprises one or more lenses, mirrors, and/or actuators for directing the fractional laser ablation output and/or the non-ablative laser coagulation output of the system to one or more desired locations on the skin of the patient. Any such lenses, mirrors, and/or actuators not inconsistent with the objectives of the present disclosure may be used. Many suitable lenses, mirrors, actuators, or other hardware or software will be readily apparent to those of ordinary skill in the art.

In addition, in some embodiments, a system described herein further comprises an imaging device configured to image a treatment area of the skin of the patient. Any imaging device or system not inconsistent with the objectives of the present disclosure may be used. Additionally, in some embodiments, the imaging device comprises both a receiver module and also a query module. A “receiver module,” for reference purposes herein, comprises one or more components configured or used to receive, detect, and/or process an imaging signal, such as a return signal (e.g., light or an acoustic return signal) provided by an imaged treatment area in response to a query by an imaging device described herein. A “query module,” for reference purposes herein, comprises one or more components configured or used to produce or emit a query, diagnostic, probe, or pilot beam that interacts with an imaging target and thereby produces a return signal from the imaging target, wherein the return signal can be used to image the imaging target. Thus, in some cases, a receiver module comprises a return signal receiver, and a query module comprises a query beam generator.

In some instances, the imaging device of a system described herein comprises a camera. In some cases, the camera is positioned or configured to receive light from the imaged treatment area, directly or through the use of one or more lenses, mirrors, or apertures. Such light can be the return signal of the imaging device. Moreover, in some embodiments, the camera can be attached to an exterior portion of a handpiece or other portion of a system described herein. Any camera not inconsistent with the objectives of the present disclosure may be used. For example, in some cases, the camera comprises a digital camera capable of capturing, recording, and/or processing two-dimensional or three-dimensional images of a target area. Further, a camera described herein can be a visible light camera or an infrared camera. Other cameras may also be used.

In other embodiments described herein, the imaging device comprises an optical imaging system, such as an optical coherence tomography (OCT) system, a multi-photon imaging system, or a reflectance confocal microscopy (RCM) system, fluorescence spectroscopy system, camera recognition and image processing system, or other optical imaging technology. As described above, such an imaging system can comprise a query module and a receiving module. For instance, in the case of an OCT imaging system, the imaging device can comprise an OCT pilot or probing beam generator and an OCT detector. The use of an OCT imaging system is especially preferred in some embodiments in which imaging beneath the surface of skin is needed or desired, such as to image a structure of skin beneath the surface. An OCT or other imaging system described herein can be used to image a component or structure of skin at any depth not inconsistent with the objectives of the present disclosure. For example, in some cases, a skin component is imaged by the imaging device at a depth of up to 2 mm, up to 1 mm, or up to 0.5 mm

In some embodiments, the imaging device of a system described herein comprises an acoustic imaging device rather than an optical imaging device. For instance, in some cases, the imaging device is an ultrasound imaging system. Such a device can comprise one or more ultrasound transducers and/or receivers.

Additionally, in some embodiments, an imaging device of a system described herein also comprises a light source (other than a laser described above). In particular, an imaging device described herein can comprise a light source for illuminating an area or surface that is to be imaged and/or treated by the system. Any light source not inconsistent with the objectives of the present disclosure may be used. For instance, in some cases, the light source comprises or is a non-laser light emitting diode or device (LED). The light source may also be an incandescent or fluorescent light bulb. Other light sources may also be used. Additionally, the light source of an imaging device described herein can be positioned or located on any portion of the imaging device or overall system not inconsistent with the objectives of the present disclosure, provided that the light source is capable of illuminating the target area.

Further, in some cases, the imaging device of a system described herein comprises computer hardware and/or software for diagnosing a condition, disease, malady, or disorder of the skin of the patient based on the image of the treatment area. Any such computer hardware and/or software not inconsistent with the objectives of the present disclosure may be used. In some instances, the computer hardware and/or software includes the hardware and/or software of a controller of the system described hereinabove. Other hardware and/or software may also be used.

Additionally, in some embodiments, a system described herein comprises a handpiece. The handpiece can have any structure not inconsistent with the objectives of the present disclosure. For example, in some cases, a handpiece of a system described herein is formed from a metal, plastic, a composite material (such as a fiber glass material), or a combination of two or more of the foregoing. The handpiece can also have an interior compartment. The interior compartment can have any size and shape not inconsistent with the objectives of the present disclosure. In some cases, the interior compartment defines, comprises, consists of, consists essentially of, or is an interior volume or region of a handpiece. Such a handpiece can be a laser treatment handpiece including a proximal end or a grip portion or member for gripping by a user of the handpiece. A handpiece can also include a distal end or head portion or member from which a laser is directed toward a target, such as a target treatment area described herein. Additionally, a handpiece described herein, in some embodiments, is attached to one or more additional components of a system described herein, such as a power source.

A handpiece described herein can also comprise an optical aperture disposed at the distal end of the interior compartment. An “optical aperture,” for reference purposes herein comprises an opening in the interior compartment that is used for the ingress and/or egress of light (such as laser light and/or light received from a target area for imaging purposes) into and/or from the interior compartment. However, it is to be understood that an “optical” aperture can also be used for the ingress and/or egress of other signals or waves, such as acoustic waves produced and/or received by an ultrasound transducer. The aperture can have any size or shape not inconsistent with the objectives of the present disclosure. In some instances, the aperture has a size sufficiently large to allow a laser beam described herein to exit the interior compartment and also sufficiently large to permit the receipt of light or another return signal from a target area for imaging purposes, including in a manner described herein. For example, in some cases, an optical aperture or opening described herein has a size in one or two dimensions (e.g., a diametrical dimension, or length and width dimensions in a plane of the opening) of up to 5 cm, up to 3 cm, up to 2 cm, up to 1 cm, up to 0.5 cm, or up to 0.1 cm. Other dimensions are also possible. Further, in some embodiments, an optical aperture described herein has a round or circular shape.

As described above, systems described herein, in some embodiments, comprise computer hardware and/or software for carrying out one or more diagnostic, imaging, and/or treatment steps described herein. Thus, in some cases, a system described herein can be at least partially automated. For example, in some cases, a system is configured to carry out an imaging, diagnosing, and/or treatment process according to instructions provided by a computer as a function of space and/or time. The computer can include a processor and a memory storing computer-readable program code portions that, in response to execution by the processor, cause instructions to be provided to one or more components of a device in a desired sequence. Any hardware and/or software not inconsistent with the objectives of the present disclosure may be incorporated into or used with a system described herein. Moreover, various suitable hardware and software components will be readily apparent to those of ordinary skill in the art. Such hardware and/or software can also be used to carry out any step or computational task not inconsistent with the objectives of the present disclosure.

Moreover, computer hardware and/or software of a device described herein can be used to direct the device to begin laser exposure (e.g., for ablation or coagulation) at essentially “the same time” as the identification/localization of target areas is ended. In other words, in some cases, imaging and treatment can occur sequentially, from a clinical perspective. For instance, in some cases, the laser exposure is begun 1 minute or less, 30 seconds or less, 20 seconds or less, 10 seconds or less, 5 seconds or less, 1 second or less, 0.5 seconds or less, or 0.1 seconds or less after the diagnosis/imaging is ended. It is also possible, in some cases, for the laser exposure to be carried out simultaneously or nearly simultaneously with the imaging/diagnosis, or partially temporally overlapping the imaging/diagnosis. Thus, in some embodiments, a system described herein enables rapid diagnosis (or imaging) and treatment of a condition, such as a skin condition, in a sequential or non-sequential manner.

Various components of systems have been described above. It is to be understood that a system described herein can include any combination of features or components described herein not inconsistent with the objectives of the present disclosure.

Some embodiments described herein are further illustrated in the following non-limiting examples.

### EXAMPLE 1

### Methods of Forming Fractional Channels

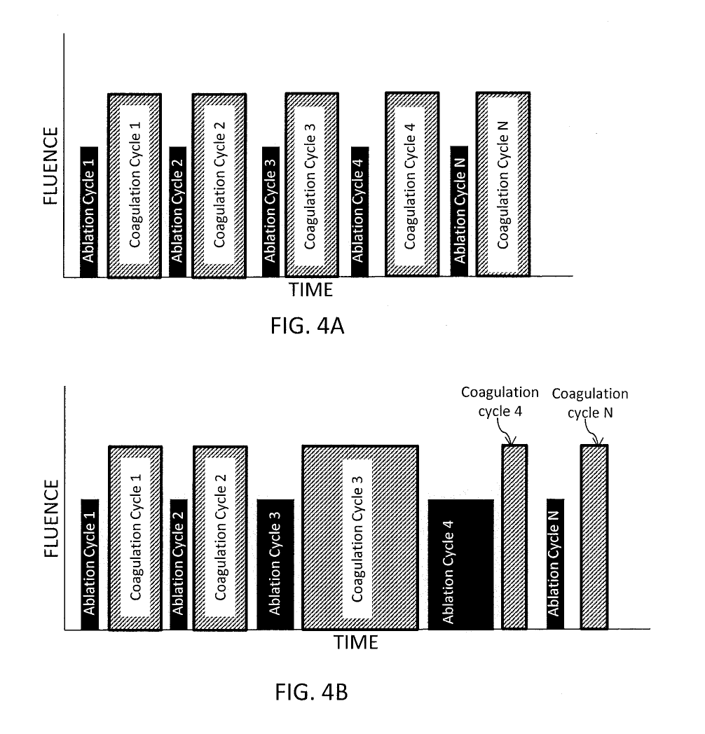

Exemplary methods of forming fractional channels according to some embodiments of methods described herein are further described with reference to FIG. 4.

FIGS. 4A-4B are graphical illustrations of exemplary ablative and coagulative laser treatments for laser treatment of tissue according to some embodiments described herein. According to FIG. 4A, alternating cycles of ablative laser light and non-ablative coagulative laser light are applied to a subject's skin. According to FIG. 4A, the ablative cycles can each be applied for a same amount of time to provide a substantially uniform channel. Similarly, each non-ablative cycle can be applied for a same amount of time to provide a substantially uniform coagulation zone.

Alternatively and as illustrated in FIG. 4B, the ablative and non-ablative cycles may be applied for a different amounts of time. Thus, the resultant ablative channel(s) and coagulation zone(s) can vary in length, width, and/or depth.

### EXAMPLE 2

### Complex Fractional Structures and Methods of Increasing Drug Uptake Using the Same

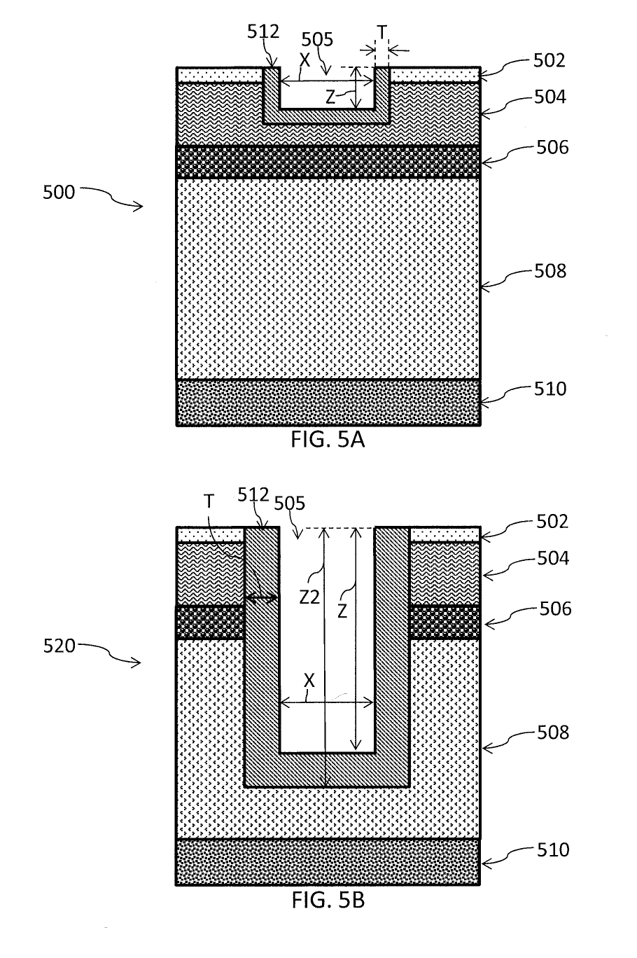

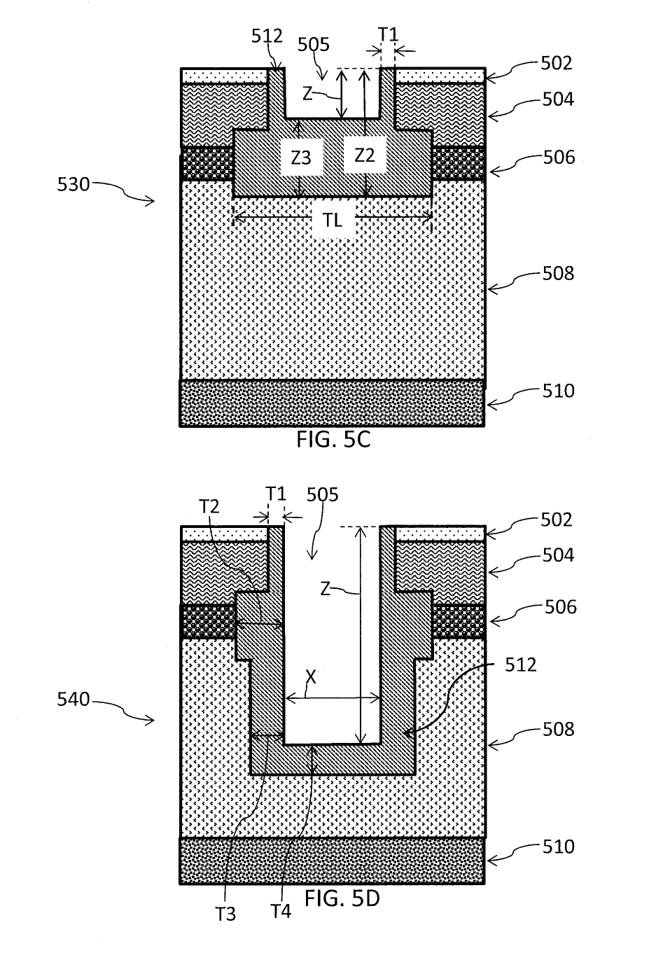

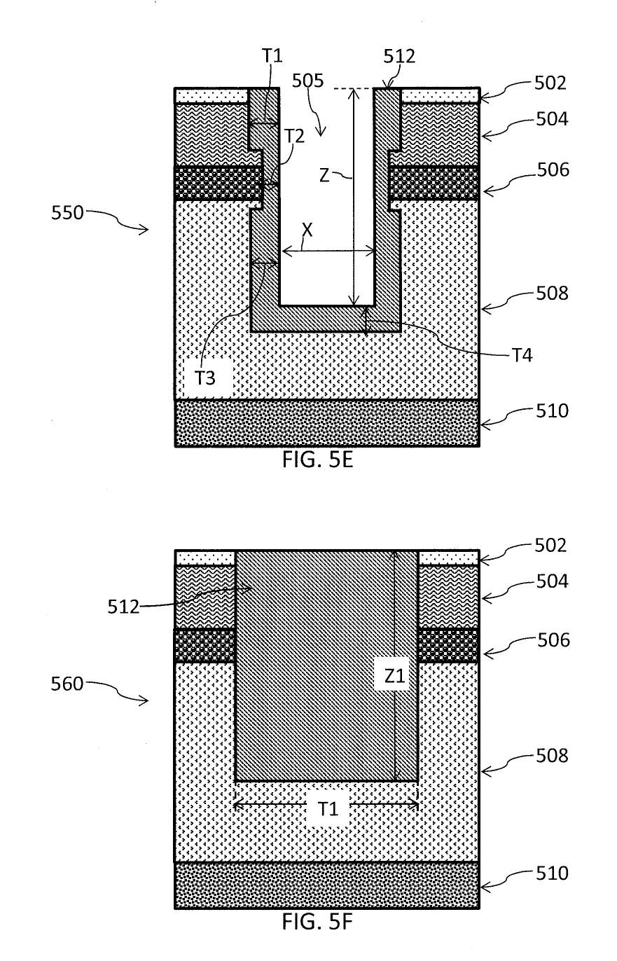

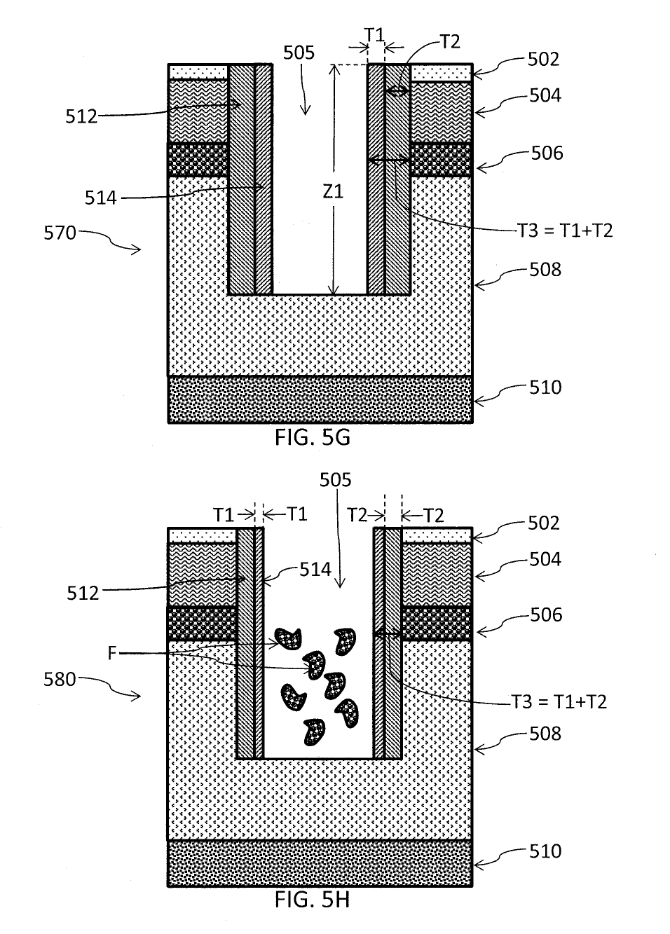

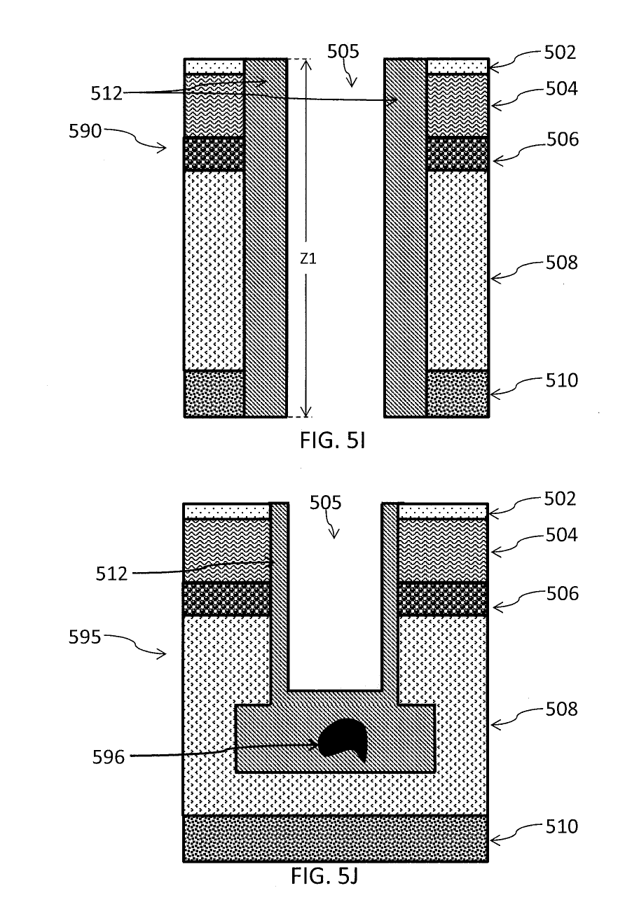

Exemplary fractional channels having complex structures according to some embodiments of methods described herein are further described with reference to FIG. 5. Additionally, the use of such exemplary structures to improve the efficacy of drug delivery in specific instances is described.

FIGS. 5A-5J schematically illustrate fractional columns (also referred to as fractional column structures) associated with combinations of ablative and non-ablative coagulative laser treatments. Each of FIGS. 5A-5J illustrates a portion of skin that includes the stratum corneum 502, the epidermis 504, the superficial vascular plexus 506, the dermis 508, and a layer of fat 510. Alternating doses of ablative and non-ablative laser light (or possibly one or more doses of ablative laser light, without non-ablative laser light) are applied to form a fractional column comprising at least one ablative channel 505 and coagulation zone 512.

In FIGS. 5A and 5B laser light has formed an ablation channel 505. The channel 505 comprises a width X, a length (not shown), and a depth Z. Each channel 505 is substantially uniform in width X and depth Z. Non-ablative laser light is used to form a coagulation zone 512 around the channel 505. As illustrated in FIG. 5A, each coagulation zone 512 has a substantially uniform thickness T. However, it is to be understood that the thickness T of the coagulation zone 512 can vary in the z-direction. Specifically, the thickness T of the coagulation zone can decrease as a function of increasing depth in the z-direction, such that the diffusion rate of a pharmaceutical composition laterally through the coagulation zone 512 increases with increasing depth. In FIG. 5A, the channel 505 in skin 500 extends into and through portions of the stratum corneum 502 and the epidermis 504. The depth of channel 505 in FIG. 5A allows for the effective uptake of pharmaceutical compositions for treating various skin conditions. The coagulation zone 512 in FIG. 5A is approximately 10-20 μm thick. More specifically, the features of the structure of FIG. 5A, particularly the width X, depth Z, and coagulation zone 512, are selected to ablate only the stratum corneum. As ablation occurs only within the upper to mid epidermis, no relevant oozing or bleeding is encountered, and the thin coagulation zone allows for effective uptake of drugs such as Levulan for the purpose of (daylight) PDT therapies.

In FIG. 5B, the channel 505 extends into and through portions of the stratum corneum 502, the epidermis 504, the vascular plexus 506, and the dermis 508. A thicker coagulation zone 512 is provided in order to seal/coagulate flows from capillary blood vessels and interstitial fluids to impede the counteractive oozing potential and to allow effective uptake of certain pharmaceutical compositions, for example, pharmaceuticals compositions that treat Basal Cell Carcinomas (BCC), such as methyl aminolaevulinate (MAL). More particularly, in certain embodiments, the coagulation zone 512 is approximately 80-100 μm thick and the channel depth Z is up to 1 mm, or about 0.8-1.2 mm, in the skin 520. For the fractional laser-mediated photodynamic therapy of certain types of BCCs, drug uptake must occur across the complete depth of the cancerous lesion, including a margin zone. A greater ablation depth and thicker coagulation zone as described above helps impede counteractive oozing potential and facilitate effective uptake of drugs. As illustrated in FIG. 5B, the coagulation zone 512 has a substantially uniform thickness T. However, it is to be understood that the thickness of the coagulation zone 512 can vary in the z-direction. Specifically, the thickness of the coagulation zone can decrease as a function of increasing depth in the z-direction, such that the diffusion rate of a pharmaceutical composition laterally through the coagulation zone 512 increases with increasing depth.

FIGS. 5C-5D illustrate non-uniform structures comprising non-uniform channels 505 and/or coagulation zones 512 that can be formed in respective portions of skin 530 and 540 after formation an initial structure, for example, after formation of the initial structure shown in FIG. 5A. In FIG. 5C, the channel 505 comprises a depth Z and an irregular coagulation zone 512 formed around portions of the channel 505. The irregular coagulation zone 512 is a global zone of thermal damage having a depth Z2 and a thickness Z3 in the z-direction. The coagulation zone 512 can comprise a first, upper portion having a first thickness T1 and a second, lower portion having a thickness TL that is greater than the first thickness T1. A further mix of ablative and non-ablative treatments can create the structure depicted in FIG. 5D, which includes a coagulation zone 512 comprising a plurality of different segments having a plurality of different thicknesses (T1-T4) extending around portions of channel 505 in the z-direction.

Again with reference to FIG. 5C and FIG. 5D, the structure depicted in FIG. 5C and FIG. 5D can be particularly suitable for a more local and targeted delivery of transdermal lidocain. In such instances, an efficient local uptake is desired, but care must also be taken to minimize entry of the pharmaceutical into the systemic circulation. Through a non-ablative wavelength, a global zone of thermal damage (depth Z2 and width TL) is first generated at the average level of the superficial vascular plexus (FIG. 5C). A further combination of ablative and non-ablative wavelengths then create the final structure having a greater final depth Z2 but thinner coagulation zone thickness (FIG. 5D). The width TL may be about 120-150 μm.

FIG. 5E illustrates skin tissue 550 having a fractional column formed therein, the fractional column comprising a substantially uniform channel 505 and a non-uniform coagulation zone 512 formed around the channel 505. The non-uniform coagulation zone 512 includes a plurality of different segments having a plurality of different thicknesses (T1-T3) disposed around the channel 505. In this embodiment, the coagulation zone 512 is thinnest proximate the vascular plexus 506. This configuration can advantageously allow a pharmaceutical composition applied to the channel 505 to migrate through the thinner portion or segment of the coagulation zone 512 and enter blood in the vascular plexus. Thus, the net flow of the pharmaceutical composition into the vascular plexus 506 can increase, and the active agent in the composition can more readily migrate into the blood stream.

FIGS. 5F and 5G illustrate formation of multiple coagulation zones 512, 514 (also referred to as first and second coagulation zones). In FIG. 5F, an initial coagulation zone 512 is Ruined in the skin 560, the coagulation zone 512 comprising a thickness T1 and a depth Z1. In FIG. 5G, a channel 505 is formed through the initial coagulation zone 512, and a second, intermediate coagulation zone 514 is formed in skin 570 via application of a non-ablative laser light. The second coagulation zone 514 is disposed between the channel 505 and initial coagulation zone 512. The initial coagulation zone 512 comprises a thickness T2 (which may be about 600 μm) and the subsequent coagulation zone 514 comprises a thickness T1 (which may be about 300 μm). This embodiment can provide for the enhanced and synergistic expression of healing and growth factors and heat shock proteins. Further, this embodiment can synergistically create a more active ecosystem for insertion of transplanted hair follicles or active (stem) cell therapies (e.g., using (human) mesenchymal stem cells).

FIG. 5H is similar to FIG. 5G, but further comprises deliberate generation of a bodily fluid F in the channel 505. The bodily fluid F can comprise blood or a component of blood (e.g., red blood cells, plasma, etc.). The deliberate generation of slight bleeding may be beneficial for accelerated healing of chronic wounds. The controlled hemorrhage may also provide nutrients and growth factors to the struggling wound surface and support the healing process. Additional healing parameters may also be applied portions of channel 505 or skin 580.

FIG. 5I illustrates skin 590 having a channel 505 and coagulation zone 512 formed therein. The channel 505 and coagulation zone 512 can comprise a depth Z1 and extend into and/or through the fat layer 510. This can provide improved drug delivery to deeper tissue, in some aspects.

FIG. 5J illustrates skin 595 having a fractional structure formed therein via multiple laser treatments for targeting, encasing, surrounding, and/or destroying a specific skin structure 596. Thermal energy generated via alternating ablative and non-ablative laser light sources can apply a customized heating profile for targeting and destroying the skin structure 596.

### EXAMPLE 3

### System for Laser Treatment

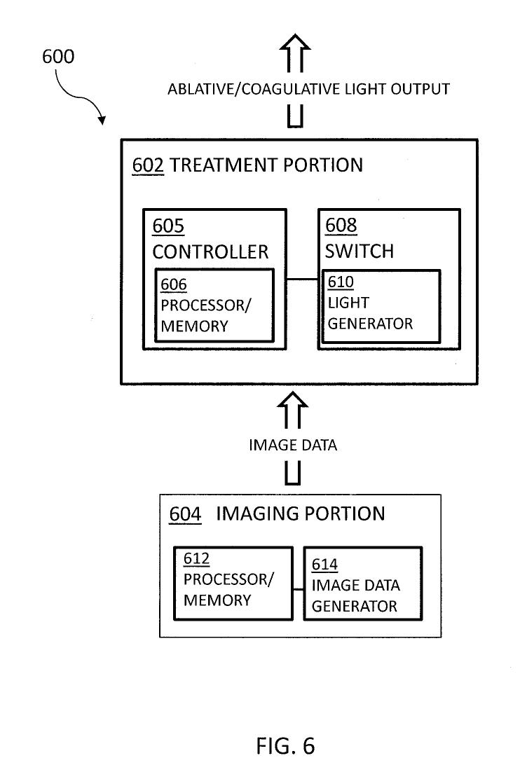

An exemplary system for laser treatment according to one embodiment described herein is further described with reference to FIG. 6.

FIG. 6 is a block diagram of an exemplary system 600 for laser treatment of skin according to some embodiments described herein. System 600 can comprise a treatment portion 602 and an imaging portion 604 (i.e., an imaging system). Treatment portion 602 can comprise a controller 605 and a switch 608. The controller 605 can receive process image data via a processor and memory 606 and instruct the switch 608 to activate a laser generator 610. The laser generator 610 can generate and switch output of the system from a laser configured to output a fractional laser ablation output to a laser configured to output a non-ablative laser coagulation output. The laser configured to perform fractional laser ablation may be the same or different than the laser configured to perform non-ablative laser coagulation. The controller 605 is configured to direct the system 600 to apply a plurality of doses of fractionally ablative laser light and a plurality of doses of non-ablative coagulative laser light to a first spot on skin of the patient.

The imaging portion 604 comprises a processor/memory 612 and an image data generator 614. The image data generator is configured to receive (e.g., as input) signals or impulses (optical, electrical, etc.) from a subject's skin, process the impulses, and generate image data. The image data can be indicative of a skin condition, disease, or a targeted skin structure (e.g., a basal cell). The image data is output to treatment portion 602 for treatment.

### EXAMPLE 4

### Methods of Treatment

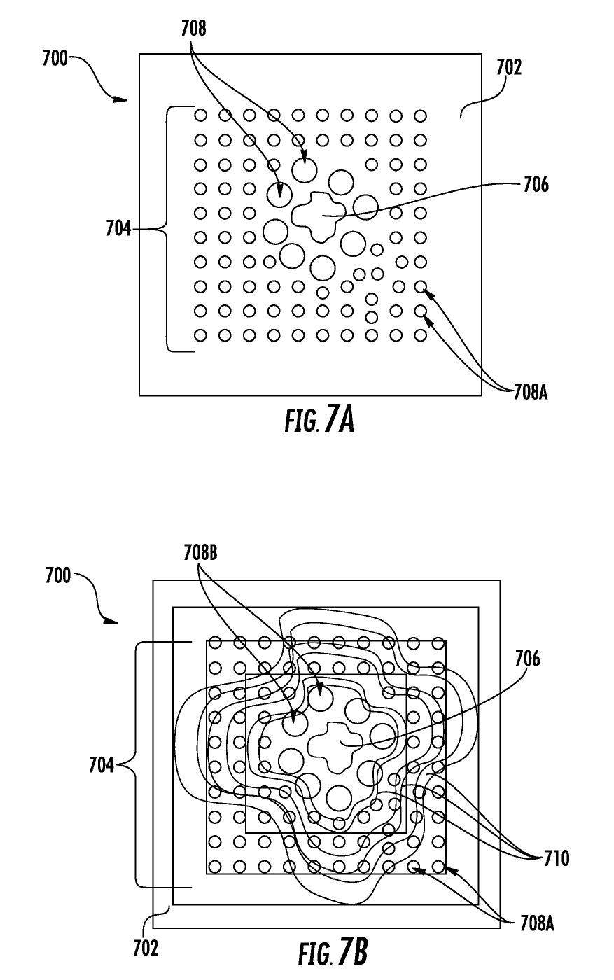

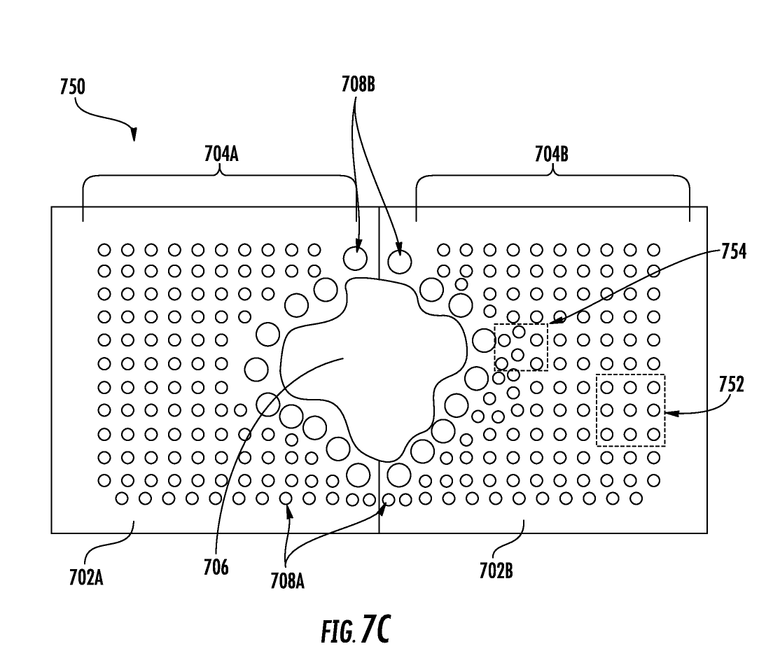

Exemplary methods for treating a patient according to some embodiments described herein are further described with reference to FIG. 7.

FIGS. 7A-7C schematically illustrate customized arrays for the combined ablative and coagulative laser treatment of tissue according to some embodiments described herein. FIG. 7A is a first array 704 customized to treat a subject's skin 700 via targeting a skin structure 706. The array 704 includes a plurality of smaller diameter smaller diameter structures 708A surrounding a plurality of larger diameter structures 708B. The larger and smaller diameter structures 708A and 708B extend into and through a surface 702 of the skin 700. The larger diameter structures 708B can surround and be disposed more proximate to portions of the targeted skin structure 706 for delivering a larger amount of pharmaceutical composition thereto. Notably, the array 704 can be customized in terms of the size(s), shape(s), number(s), and/or placement of the various structures (e.g., 708A, 708B). Each customized array may include multiple different sizes, shapes, and/or depths of structures (i.e., 708A, 708B).

FIG. 7B illustrates the heating profile generated via the multi-structured array 704 when applied to skin 700. The heating profile can comprise a plurality of concentric thermal waves surrounding the skin target 706.

FIG. 7C schematically illustrates customized arrays for the combined ablative and coagulative laser treatment of skin 750. The customized arrays may be disposed over multiple treatment windows 702A, 702B. A first array 704A over the first window 702A can abut a second array 704B over the second window 702B. A targeted skin structure 706 can be disposed between portions of opposing windows 702A, 702B. Each array 704A, 704B includes a plurality of smaller diameter smaller diameter structures 708A surrounding a plurality of larger diameter structures 708B. The structures can be spaced apart at regular or random intervals. For example, are 752 includes a regular 3×3 array of structures. Area 754 includes an irregular array of randomly spaced structures. Arrays set further herein can comprise any size, shape, quantity, and/or spatial arrangement of structures consistent with the instant disclosure, for providing areas of skin having various structures that facilitate customized cutaneous drug delivery.

1. A method of laser treatment, the method comprising:
forming at least one fractional column of tissue in skin of a patient,
wherein the fractional column has a structure along a z-direction orthogonal to an exterior surface of the skin; and
wherein the structure of the fractional column varies along the z-direction in one or more of:
angular orientation relative to the exterior surface of the skin;
ablated channel width;
coagulation zone thickness;
coagulation zone offset in an x-direction or a y-direction perpendicular to the z-direction;
coagulation zone intensity; and
thermal insult.

2. The method of claim 1, wherein the structure of the fractional column along the z-direction is defined by a plurality of segments that differ in one or more of the angular orientation relative to the exterior surface of the skin, ablated channel width, coagulation zone thickness, coagulation zone offset in an x-direction or a y-direction perpendicular to the z-direction, coagulation zone intensity, and thermal insult.

3-8. (canceled)

9. The method of claim 2, wherein the plurality of segments define a step function with respect to one or more of the angular orientation relative to the exterior surface of the skin, ablated channel width, coagulation zone thickness, coagulation zone offset in an x-direction or a y-direction perpendicular to the z-direction, coagulation zone intensity, and thermal insult.

10. The method of claim 2, wherein the plurality of segments define a continuous function with respect to one or more of the angular orientation relative to the exterior surface of the skin, ablated channel width, coagulation zone thickness, coagulation zone offset in an x-direction or a y-direction perpendicular to the z-direction, coagulation zone intensity, and thermal insult.

11. The method of claim 2, wherein at least two segments of the plurality of segments have different molecular diffusion rates and/or different temperature gradients in a lateral direction orthogonal to the z-direction.

12. The method of claim 1, wherein forming the at least one fractional column comprises applying a plurality of doses of fractionally ablative laser light to the skin of the patient.

13. The method of claim 1, wherein forming the at least one fractional column comprises applying one or more doses of non-ablative coagulative laser light to the skin of the patient.

14. The method of claim 1, wherein forming the at least one fractional column comprises applying a plurality of doses of fractionally ablative laser light and a plurality of doses of non-ablative coagulative laser light to a first spot on the skin of the patient.

15. The method of claim 1 further comprising:
imaging a treatment area of the skin of the patient to obtain an image of the treatment area,
wherein forming the at least one fractional column in the skin of the patient comprises forming the at least one fractional column within the treatment area of the skin of the patient, and
wherein imaging the treatment area is carried out before forming the at least one fractional column in the skin of the patient.

16. (canceled)

17. The method of claim 15 further comprising:
diagnosing a condition of the skin of the patient based on the image of the treatment area, before forming the at least one fractional column in the skin of the patient.

18. The method of claim 17 further comprising:
determining one or more features of the at least one fractional column based on the diagnosed condition of the skin, before forming the at least one fractional column in the skin of the patient.

19. The method of claim 1, wherein a plurality of fractional columns of tissue is formed in the skin of the patient.

20-27. (canceled)

28. The method of claim 19 further comprising:
imaging a treatment area of the skin of the patient to obtain an image of the treatment area,
wherein forming the plurality of fractional columns in the skin of the patient comprises forming the plurality of fractional columns within the treatment area of the skin of the patient, and
wherein imaging the treatment area is carried out before forming at least one of the plurality of fractional columns in the skin of the patient.

29. (canceled)

30. The method of claim 28 further comprising:
diagnosing a condition of the skin of the patient based on the image of the treatment area, before forming the plurality of fractional columns in the skin of the patient.

31. (canceled)

32. The method of claim 30 further comprising:
determining an order of forming the plurality of fractional columns based on the diagnosed condition of the skin, before forming the plurality of fractional columns in the skin of the patient.

33. The method of claim 1 further comprising:
applying a pharmaceutical composition to the exterior surface of the skin.

34-36. (canceled)

37. A method of increasing the uptake of a pharmaceutical composition by a patient, the method comprising:
applying the pharmaceutical composition to an exterior surface of skin of the patient; and
forming at least one fractional column of tissue in the skin of the patient,
wherein the fractional column has a structure along a z-direction orthogonal to an exterior surface of the skin; and
wherein the structure of the fractional column varies along the z-direction in one or more of:
angular orientation relative to the exterior surface of the skin;
ablated channel width;
coagulation zone thickness;
coagulation zone offset in an x-direction or a y-direction perpendicular to the z-direction;
coagulation zone intensity; and
thermal insult.

38-42. (canceled)

43. A system for laser treatment of a patient, the system comprising:
a laser configured to perform fractional laser ablation;
a laser configured to perform non-ablative laser coagulation;
a switching component configured to switch output of the system from a fractional laser ablation output to a non-ablative laser coagulation output;
a controller configured to direct the system to apply a plurality of doses of fractionally ablative laser light and a plurality of doses of non-ablative coagulative laser light to a first spot on skin of the patient.

44. The system of claim 43, wherein a single laser is configured to selectively perform fractional laser ablation and non-ablative laser coagulation.

45. The system of claim 43, wherein the laser configured to perform fractional laser ablation is a first laser and the laser configured to perform non-ablative laser coagulation is a second laser, the first and second lasers differing from one another.

46-54. (canceled)
